(19) United States
(12) Patent Application Publication
(10) Pub. No.: US 2017/0216148 A1
(43) Pub. Date: Aug. 3, 2017

(54) SYSTEM AND METHOD FOR SALIVA REPLENISHMENT AND CONTROL

(71) Applicant: Jennifer Rebecca Altounian, White Lake, MI (US)

(72) Inventor: Jennifer Rebecca Altounian, White Lake, MI (US)

(21) Appl. No.: 15/373,555

(22) Filed: Dec. 9, 2016

Related U.S. Application Data
(60) Provisional application No. 62/265,172, filed on Dec. 9, 2015.

(51) Int. Cl. A61J 7/00; A61M 1/00; A61M 31/00

(52) U.S. Cl. CPC A61J 7/0053; A61M 31/00; A61M 1/0058; A61M 2210/0625

(57) ABSTRACT

Disclosed is a system for providing artificial saliva. The system can include the mouthpiece, a portable supply unit, and a stationary supply unit. The portable supply unit can be adapted to be carried by a user. The portable supply unit can include a first fluid system, a first interface module, and a first control module. The first fluid system can be adapted to fluidly couple to the mouthpiece.

### CROSS-REFERENCE TO RELATED APPLICATIONS

This application claims the benefit of U.S. Provisional Application No. 62/265,172, filed on Dec. 9, 2015. The entire disclosure of the above application is incorporated herein by reference.

### FIELD

The present disclosure relates to systems and methods for saliva replenishment and control and, more particularly, to mouthpieces and fluid delivery systems.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Saliva production and removal is important to several biological functions including swallowing, digestion, and oral hygiene. For example, enzymes in saliva aid digestion. As another example, saliva hydrates the mouth and throat, aiding swallowing and promoting oral hygiene. Salivary glands may become impaired and/or damaged due to accident, trauma, illness, disease, and/or medical treatments including surgery, medications, radiation therapy, and chemotherapy treatments. In some cases, the accident, trauma, illness, disease, or medical treatment can seriously impair or permanently damage the salivary glands and, as a consequence, render normal salivary production impossible.

As one example, a condition referred to as Xerostomia in the medical community afflicts millions of people worldwide. Xerostomia is used to refer to a condition in which saliva production is impaired or absent. Xerostomia can be caused by various diseases such as Sjögren's syndrome, human immunodeficiency virus (HIV), Alzheimer's disease, diabetes, cystic fibrosis, lupus, and rheumatoid arthritis. As another example, people undergoing cancer treatments such as radiation therapy and chemotherapy to the head and neck often experience a loss in saliva production, and the loss can be permanent. Medications may also diminish saliva production. Saliva production may also diminish as an individual ages and can become problematic at advanced ages.

Several problems can arise in an individual whose saliva production or removal is compromised. Without the ability to produce or remove saliva, an individual may not properly produce the enzymes necessary to properly digest food. Tooth decay, painful sores in the mouth, problems swallowing, and the inability to eat and speak can arise and can cause other significant health issues. For example, psychological stress and/or other problems can develop. At a minimum, an individual's quality of life can be negatively impacted.

Accordingly, there is a need for devices that can be used to replenish and remove saliva and control saliva production, as well as provide a more convenient way to deliver medications to patients. Several devices have been developed. However, these devices can be improved upon. Specifically, the comfort, versatility, operation, and appearance can be improved upon. Through such improvements, patient compliance with therapies directed to saliva production and other health issues can be improved along with the patient's quality of life. Additionally, such improvements can make certain treatments such as cancer treatments more tolerable and provide for a more stable overall health.

### SUMMARY

In one form, the present disclosure provides a mouthpiece. The mouthpiece can include a hydrophilic foam surrounded by a heat shrinkable layer. The hydrophilic foam member can be adapted to engage a lower dental arch adjacent a first tooth on a first side of a mouth. The hydrophilic foam member can include a first passage extending between a vestibule on the first side of the mouth. Optionally, an anchoring member can be adapted to engage the lower dental arch or a patients lip. The first tube can extend within the mouth cavity proper from the hydrophilic foam member adjacent to an inner gum line. The second tube can extend within the vestibule on the first side of the mouth adjacent to an outer gum line. The second tube can include a first end fluidly coupled to the first passage and a second end extending outside of the mouth.

The present disclosure also provides a system that can include the mouthpiece, a portable supply or removal unit, and a stationary supply unit. The portable supply or removal unit can be adapted to be carried by a user. The portable supply or removal unit can include a first fluid system, a first interface module, and a first control module. The first fluid system can be adapted to fluidly couple to the mouthpiece. The first fluid system can supply or remove saliva replenishment fluid to the mouth via the mouthpiece and/or remove fluid from the mouth via the mouthpiece based on first operational settings. The first interface module can receive a first input and can communicate the first operational settings with the stationary supply unit. The first control module can selectively adjust the first operational settings based on the first input and second operational settings communicated by the stationary supply unit. The stationary supply unit can include a second fluid system, a second interface module, and a second control module. The second fluid system can be adapted to fluidly couple to the mouthpiece. The second fluid system can supply saliva replenishment fluid to the mouth via the mouthpiece and/or remove fluid from the mouth via the mouthpiece based on the second operational settings. The second interface module can receive a second input and can communicate the second operational settings with the portable supply unit. The second control module can selectively adjust the second operational settings based on the second input and the first operational settings.

In another form, the present disclosure provides methods related to a mouthpiece and a system according to the present disclosure.

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIGS. 1a and 1b are an environmental perspective view a first exemplary mouthpiece according to the present disclosure;

FIG. 2 is an environmental perspective view of the mouthpiece shown in FIGS. 1a and 1b;

FIG. 3 is an exploded view of the mouthpiece shown in FIG. 2;

FIGS. 4a and 4b are illustrating an exemplary tube used in the mouthpiece shown in FIGS. 1a-3;

FIG. 5 is an optional stiffening member used in the mouthpiece shown in FIGS. 1a-3;

FIGS. 6a-c represent a suction tube associated with the mouthpiece shown in FIGS. 1a-3;

FIGS. 7a-7d represent an alternate upper and lower saliva replenishment prosthesis according to the present teachings;

FIG. 8 represents the installation of a lower mouth prosthetic according to the present teachings;

FIGS. 9a-9c represent an alternate upper saliva replenishment prosthesis according to the present teachings;

FIGS. 10a-10d represent an alternate upper saliva replenishment prosthesis according to the present teachings;

FIGS. 11a-11j represent an alternate saliva replenishment prosthesis according to the present teachings;

FIGS. 12a-12f represent over the tooth saliva replenishment prosthesis according to the present teachings; and

FIGS. 13a-13d represent alternate saliva replenishment prosthesis according to the present teachings.

### DETAILED DESCRIPTION

The following description is merely illustrative in nature and is in no way intended to limit the disclosure, its application, or uses. For purposes of clarity, the same reference numbers will be used in the drawings to identify similar elements. As used herein, the phrase at least one of A, B, and C should be construed to mean a logical (A or B or C), using a non-exclusive logical or. It should be understood that steps within a method may be executed in different order without altering the principles of the present disclosure.

The present disclosure provides a mouthpiece that can be attached to a lower portion of a mouth and can be used to replenish or remove and/or control saliva in a patient or user. The user can be a male or female and can be an adult or child. The mouthpiece can be used during treatment of temporary conditions such as a temporary loss of swallowing capability due to an accident or trauma, or chronic conditions and diseases such as Xerostomia and cancers affecting salivary function. The mouthpiece can be used to supply and/or remove fluid from the mouth. The mouthpiece is designed so that it can be relatively inconspicuous and minimally intrusive, and can be continuously worn for prolonged periods, for example days and weeks. The mouthpiece is further designed so that it can be used to supply a saliva replenishment fluid in a manner that mimics a normal saliva flow within the mouth. By incorporating such design features, the mouthpiece can be comfortably used without compromising chewing, eating, speaking, and sleeping, and can promote patient compliance with therapies dependent on the use of the mouthpiece.

In various aspects, the mouthpiece can be individually sized and made to fit a particular user. The mouthpiece can have a modular construction that further enables components of the mouthpiece as produced to be altered and custom fit to a particular patient. The modular construction can also enable one or more components of the mouthpiece to be individually replaced without the need for another complete replacement mouthpiece. The components can be individually replaced to maintain a desired sanitary condition of the mouthpiece.

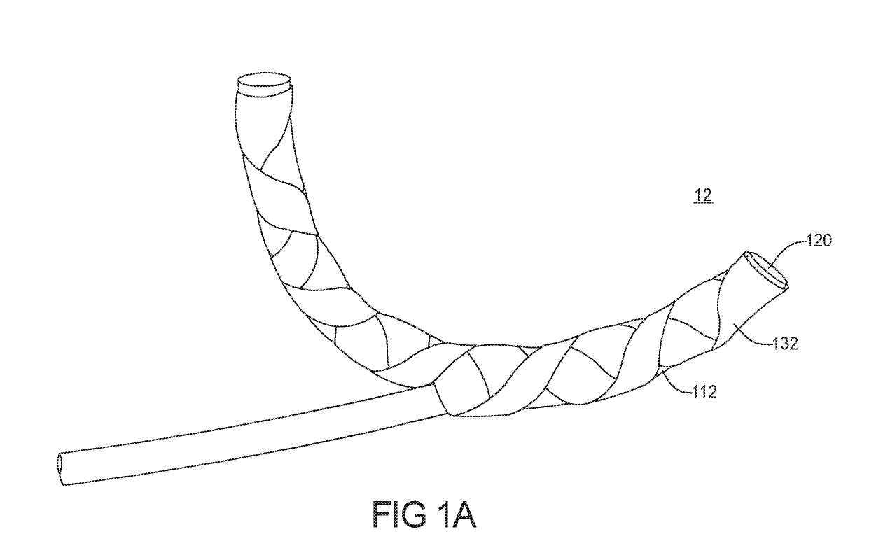

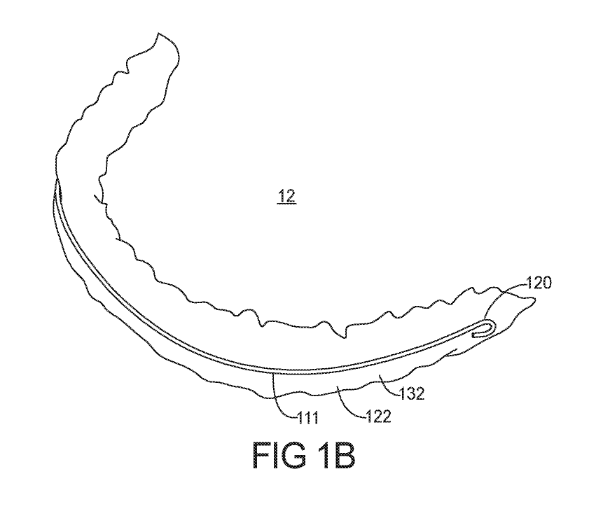

With particular reference to FIGS. 1a-3, environmental views of a lower portion of an exemplary mouthpiece 12 according to the present disclosure. The mouthpiece 12 can be anchored adjacent the lower teeth 20 anchored in a lower jaw bone by gingiva or gum. The lower dental arch includes alveolar processes that receive roots of the lower teeth, the lower teeth, and portions of the gum covering the alveolar processes and surrounding the lower teeth. The lower teeth can include up to twelve deciduous teeth in a child and up to permanent teeth in an adult. For exemplary purposes, the lower teeth presented include twelve teeth: four incisors, two canines, four premolars, and two molars. The lower teeth include lingual surfaces facing the mouth cavity proper and a tongue (not shown), labial or buccal surfaces facing the vestibule 34 and a cheek (not shown) and lips (not shown), and surfaces of contact between adjoining teeth. The lower teeth further include crowns facing upper teeth of an upper jaw bone and defining a lower bite surface or plane. Together, the lower teeth and the gum define an inner gum line and an outer gum line. Openings of a submandibular duct within the mouth cavity proper, which may be referred to as the Wharton's duct, are illustrated by openings.

The mouthpiece 12 can include a first foam anchoring member 100, a second member 102 annularly disposed about the first foam anchoring member 100, a first tubular member 104 partially disposed within the first foam anchoring member 100 and supported by the second member 102. The first and second anchoring members 100 and 102 can be sized to fit within the spaces between the lower lip and the lower teeth, with the first tubular member protruding from the mouth the provide vacuum or saliva substitute to the mouth. Optionally, the mouthpiece can include a deformable first foam anchoring member 100 can be generally solid structures and can have various shapes adapted to fit within the spaces adjacent to the molars and adjoining teeth of the lower teeth.

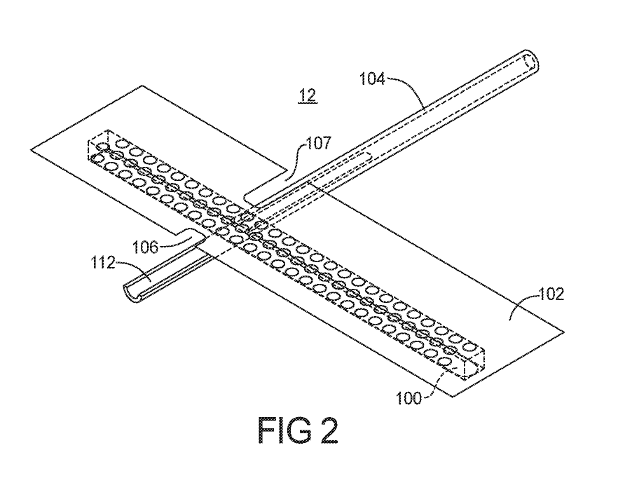

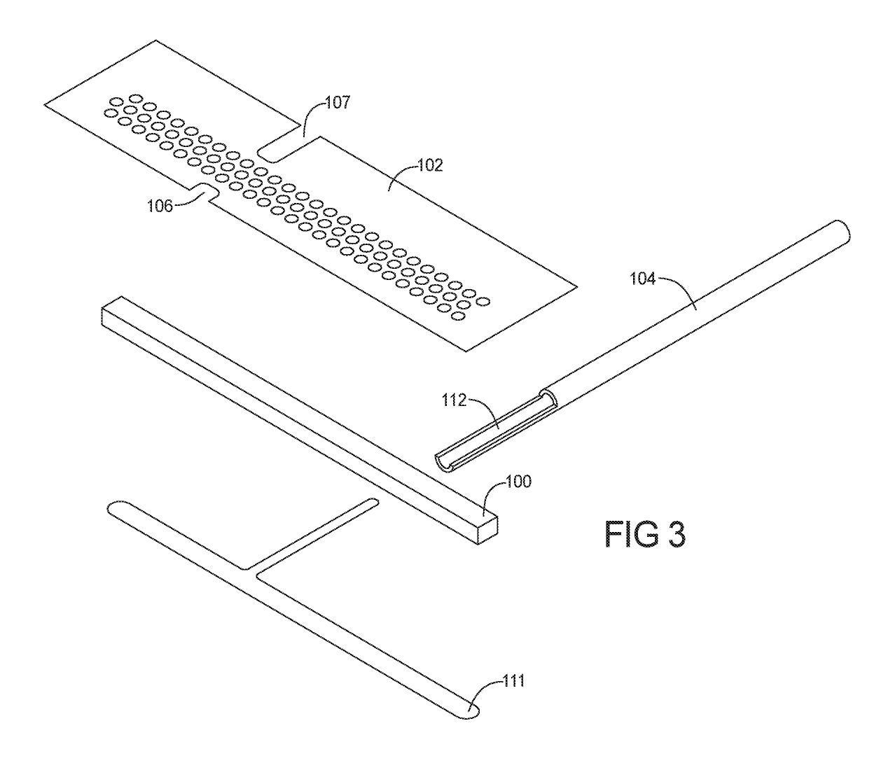

With additional reference to FIGS. 2 and 3, the mouth piece 12 can include a foam member 120, an anchoring tube 122 (shown here flattened), and the first tubular member 104. The hydrophilic foam member 120 extends about the lower gum. The hydrophilic foam member 120 can have a generally tapered polyhedral shape as illustrated, or can have a contoured shape resembling a natural tooth that may otherwise reside in the space.

The foam member 100 can be Hydrasorb® Foam Hydrasorb® the name of a group of medical-grade polyurethane, hydrophilic foams. This foam is manufactured from a base material of polyether polyisocynate resins. Hydrasorb® is sterilizable. And can be Die cut or 18″×36″ sheets (⅛″ to ¾″ wetted thickness) or molded to shape. Hydrophilic Absorption Capacity (Water): up to 15×Dry Wt. [ASTM D1667] Cell Structure (DRY Avg.): 86 Cells/Linear In. Density (Nominal/DRY): 7.5 lb/ft3 [ASTM D3574] Elongation % (DRY Avg.): 650% [ASTM D3574] Expansion in Water (length)(Avg.): 31% (Avg.) [ASTM F1087] Foam Moisture Content (DRY Avg.): 3.56% [Karl Fischer Method] Indention Force Deflection (IDF): [ASTM D3574 Test B1] IDF @ 25%: (N) 133% IDF @ 65%: (N) 346% Resiliency/Rebound Test (RT [ASTM D3574 Test B1] RT @ 25%: (N) 121% Compression Set (DRY Avg.): [ASTM D3574] 25%: 16.0% 50%: 36.0% Tensile Strength (Dry): 30.0 lbf/In2 [ASTM D3574 Test B1]

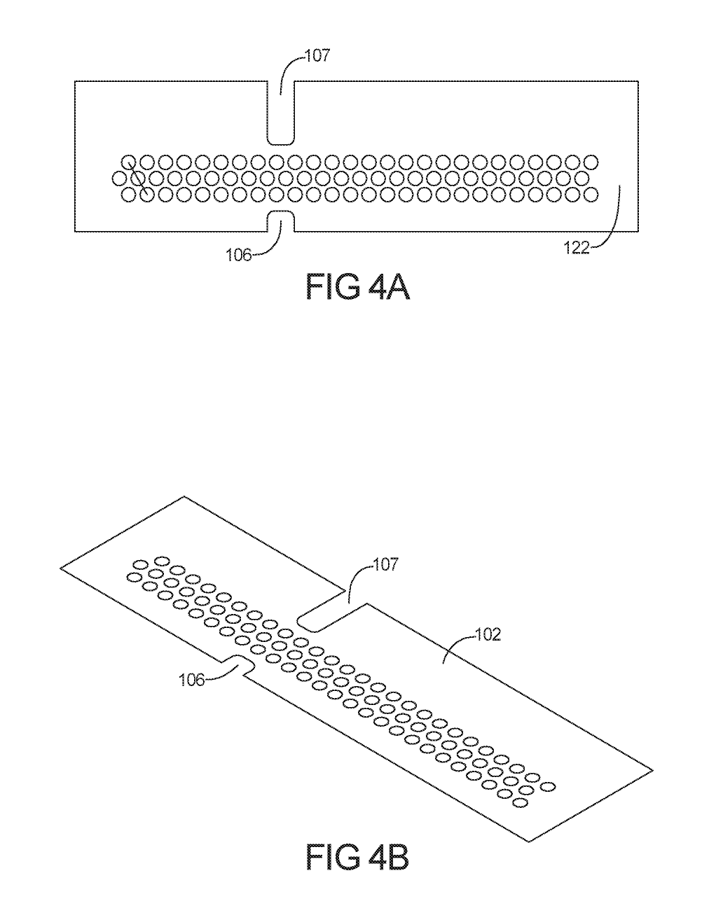

As shown in FIGS. 4a and 4b, the anchoring tube 122 is preferably perforated two accept saliva from the mouth or saliva replacement tube 104. The size and shapes of these perforations can vary. In a flattened configuration, a notch or pair of notches 106, 107 are utilized to be positioned about the tube 104 and the hydrophilic foam 102.

The front surface 132 can be adapted and disposed to engage one or more of the surface of contact, the lingual surface, and the buccal surface of the molar. The front surface 132 can be further disposed to allow one end of the tube 104 to exit the front surface 132 adjacent the lingual surface of the molar and an opposite end of the passage to exit the front surface adjacent the buccal surface of the molar. In this way, the front surface 132 can be disposed to allow the first tubular member 104 to extend from the front surface 132 adjacent the lingual surface of the molar, and the second tubular member to extend from the front surface adjacent the buccal surface 62 of the molar. The front surface 132 can be generally flat as illustrated by the present example and, optionally, can include a portion complementary to the adjoining surface of contact of the molar. In this way, the front surface can engage and thereby resist relative movement between the first foam anchoring member 100 and the molar 56.

In various aspects, the first and second anchoring members 100 and 102 can be made in a mirror image to that described above. In this way, the mouthpiece 12 may be configured so that the tubular member 104 exits the mouth 10 on the left side of the user.

In various aspects, the first and second anchoring members 100 and 102 can be attached in any suitable manner. For example, a suitable adhesive such as an adhesive that adheres dentures to gum may be used. In various aspects, the first and second anchoring members 100 and 102 can be attached in a semi-permanent manner using a bone fastener. In various aspects, the first and second anchoring members 100 and 102 can be made from any suitable dental material which allows saliva infiltration. Suitable dental materials include, but are not limited to, biocompatible polymers such as acrylic materials, and metals such as titanium.

FIG. 4 represents a metal deformable support member 111 which can be inserted adjacent to the hydrophilic foam member 100. This metal deformable support member can then be used to form the mouthpiece prior to insertion between the lip and gum adjacent to the lower teeth.

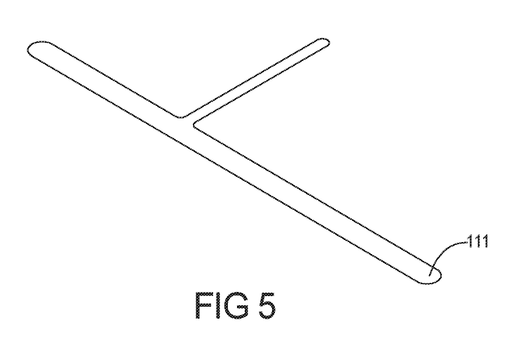

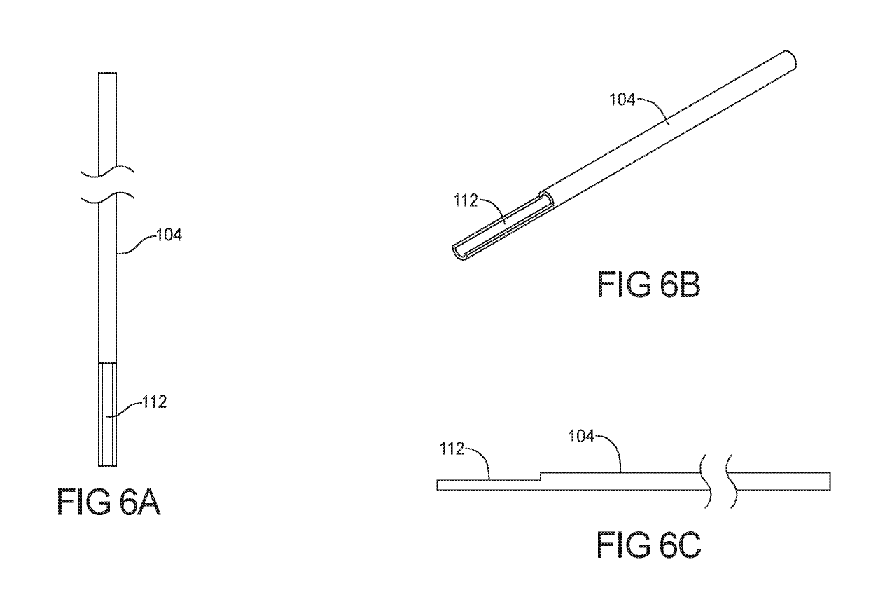

FIGS. 6a-6c represent tubes which are placed adjacent to the hydrophilic foam member and within the heat shrink material of tube 102. As described about, a portion of the tube is passed though the slots 106 and 107. The tube 104 has a first portion which is perforated or notched 112 to allow suction to be applied to the open pore hydrophilic foam 100.

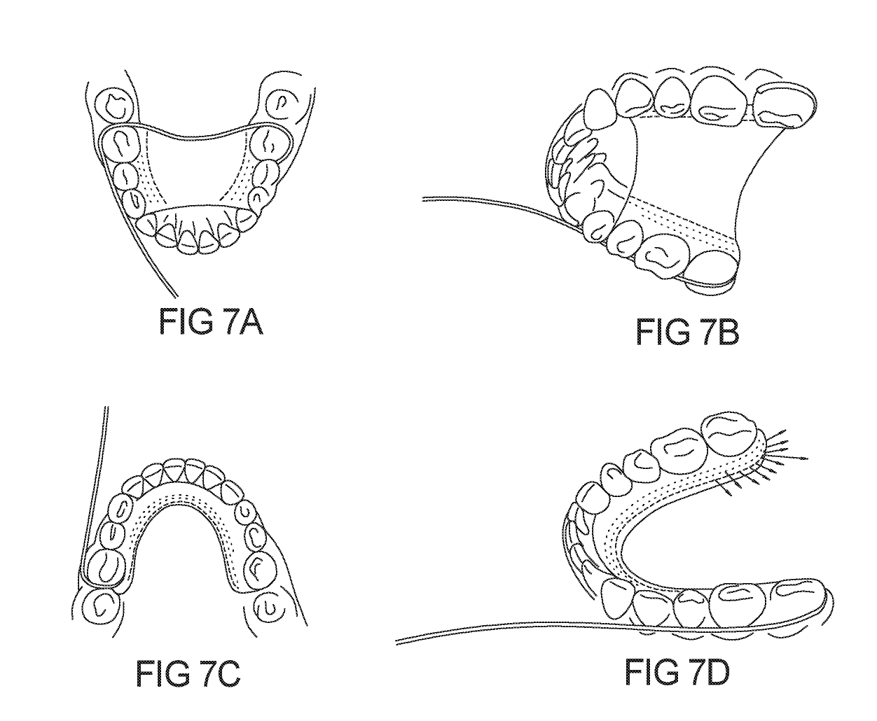

FIGS. 7a-7d represent an alternate upper and lower saliva replenishment prosthesis according to the present teachings. The upper and lower prosthesis are made of materials having a soft durometer. They define an inner passage and a plurality of apertures which link an outer surface of the prostheses with the inner aperture. As can be seen, each prosthesis utilizes an outer supply or extraction tube which can be coupled to a fluid supply or a vacuum as described above.

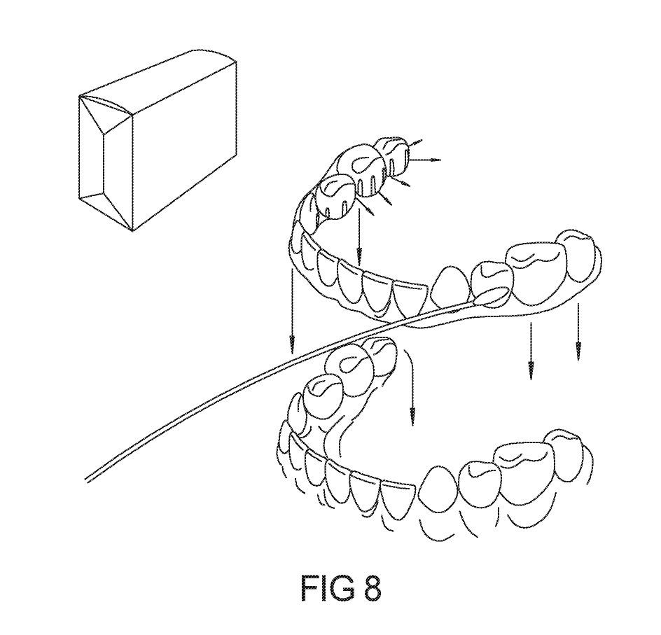

FIG. 8 represents the installation of a lower mouth prosthetic according to the present teachings. Shown is a film layer, which is coupled to the teeth using a water soluble adhesive. The fluid extraction tube is placed along the outside of the tooth ridge. Should a vacuum be drawn through the tube, fluid is drawn through apertures defined through the polymer layer. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

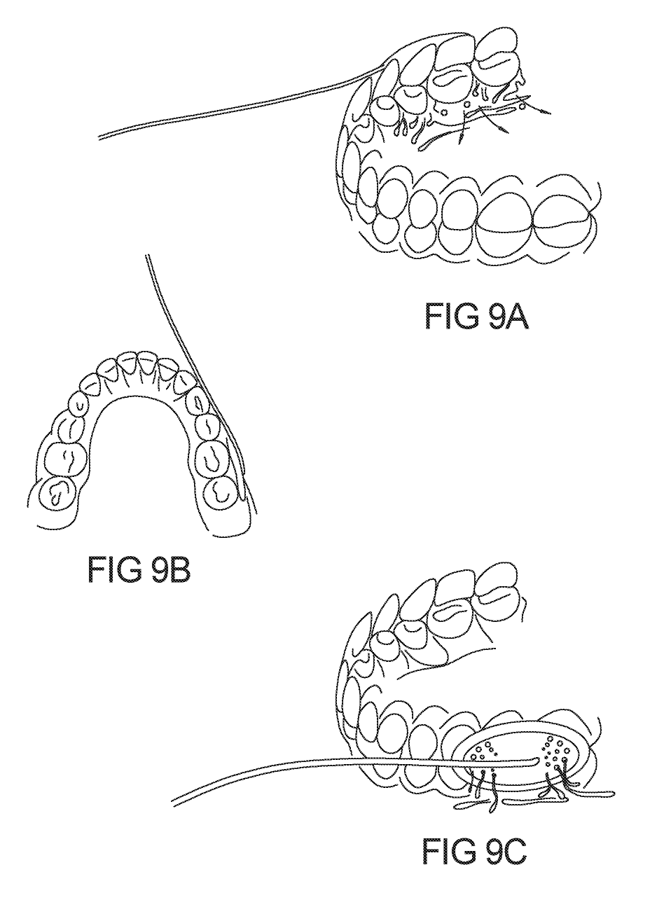

FIGS. 9a-9c represent an alternate upper saliva replenishment prosthesis according to the present teachings. As is shown, a deformable polymer material has a fluid transport tube disposed therethrough. The deformable member is generally oval and is configured to be fixed to the molars between the tooth and the gum line. The device has a through passage and a plurality of excretion or vacuum holes. The fluid extraction tube is placed along the outside of the tooth ridge. Should a vacuum be drawn through the tube, fluid is drawn through apertures defined through the polymer layer. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

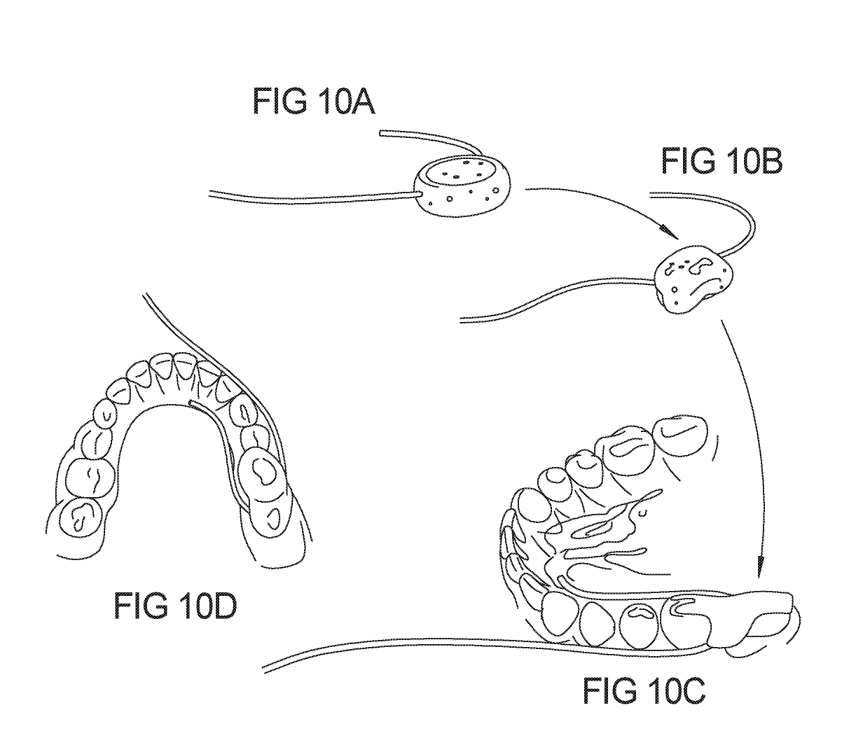

FIGS. 10a-10d represent an alternate saliva replenishment or removal prosthesis according to the present teachings. The fluid extraction tube is placed along the outside of the tooth ridge. The material is plastically deformable an as a plurality of through passages which remain open upon disposition over the teeth. They passages remain open because they have a surface which resists the sticking of one inner aperture surface to another. They are also configured to be strong enough not to collapse of the application of the vacuum. Should a vacuum be drawn through the tube, fluid is drawn through apertures defined through the polymer material. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

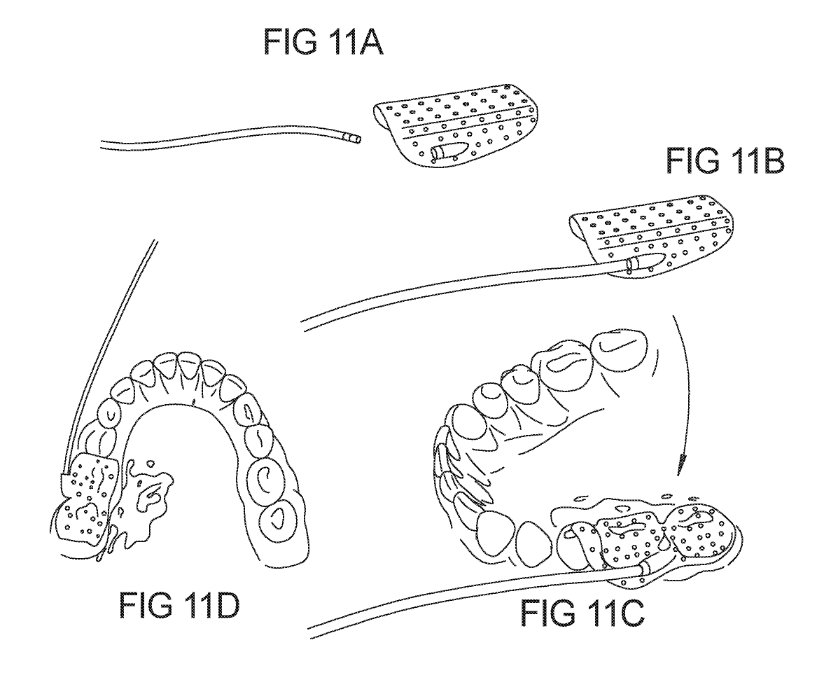

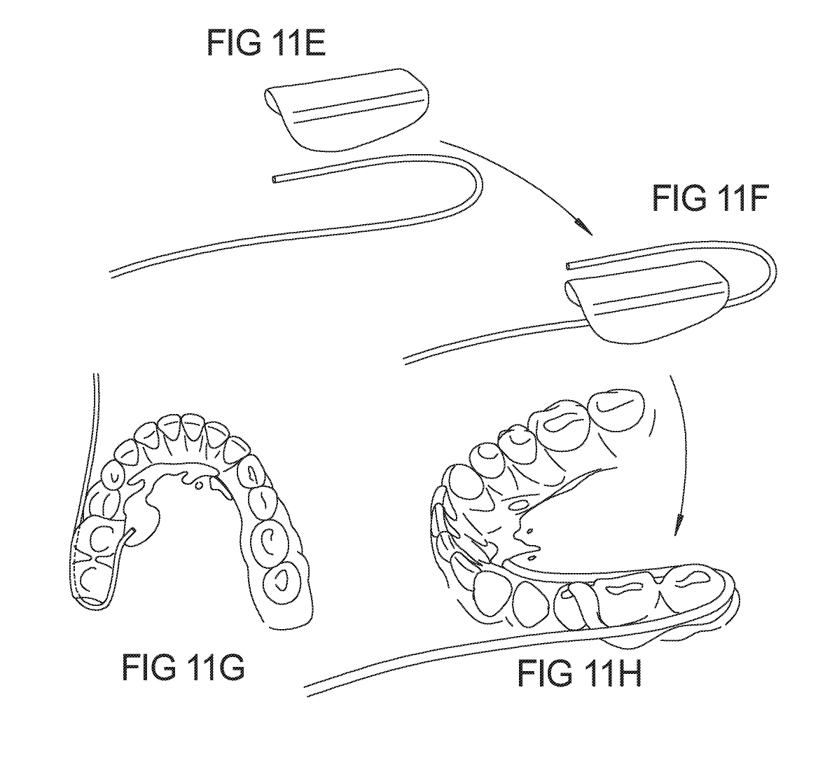

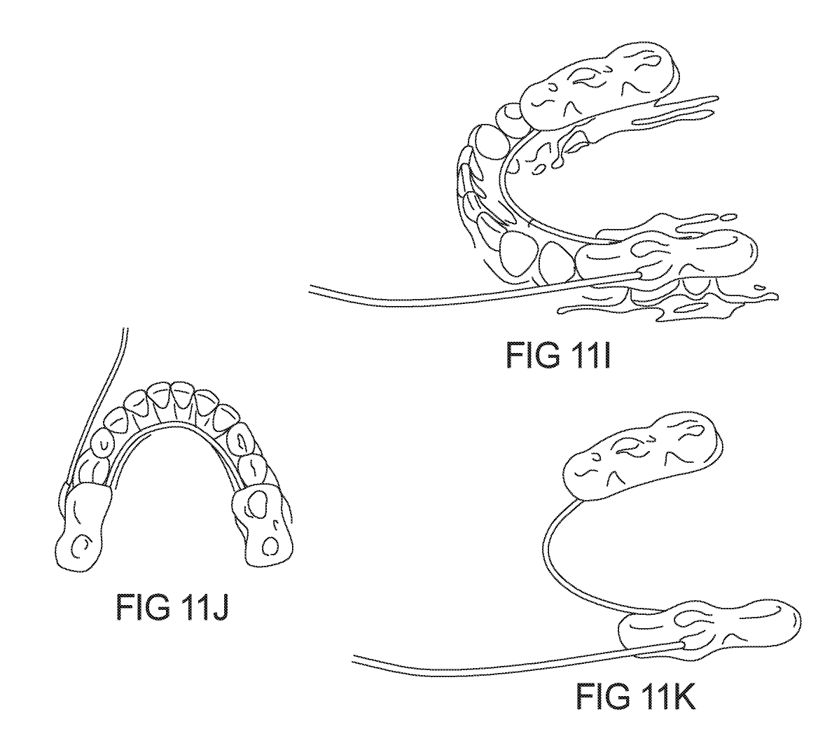

FIGS. 11a-11j represent an alternate saliva replenishment prosthesis according to the present teachings. As is shown, a deformable polymer material has a fluid transport tube disposed therethrough. The deformable member is generally oval and is configured to be fixed to the molars between the tooth and the gum line. The device has a through passage and a plurality of excretion or vacuum holes. The fluid extraction tube is placed along the outside of the tooth ridge. Should a vacuum be drawn through the tube, fluid is drawn through apertures defined through the polymer layer. This configuration can be used as both the upper and lower prosthesis are made of materials having a soft durometer. They define an inner passages between the teeth which can us used to draw out saliva. The plurality of apertures link an outer surface of the prostheses with the inner aperture. As can be seen, each prosthesis utilizes an outer supply or extraction tube which can be coupled to a fluid supply or a vacuum as described above.

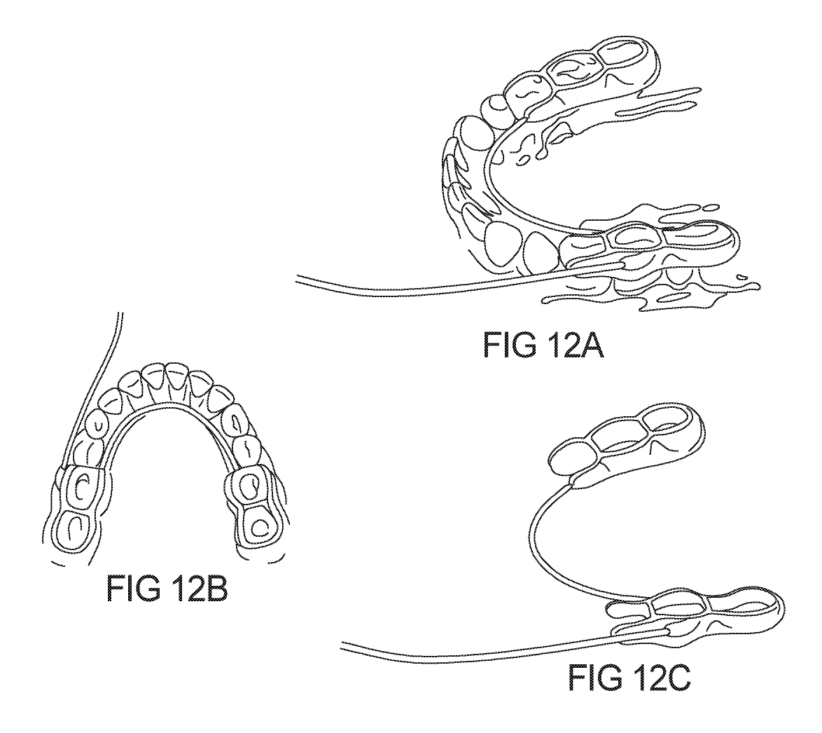

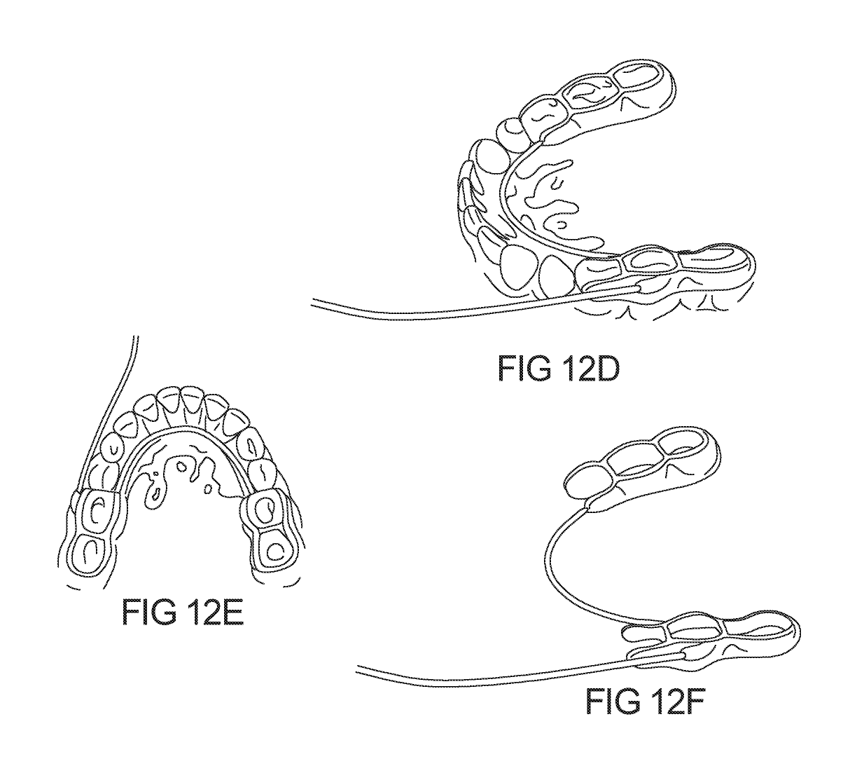

FIGS. 12a-12f represent an alternate saliva replenishment prosthesis according to the present teachings. The prosthetic member is formed around the molars and do not interfere with the molar bite surface. Similar to the teachings in FIGS. 7a-7d, alternate upper and lower saliva replenishment prosthesis according to the present teachings. As can be seen the prosthesis travels over the molar region and is positioned under the tongue at only a single location. The upper and lower prosthesis are made of materials having a soft durometer. They define an inner passage and a plurality of apertures which link an outer surface of the prostheses with the inner aperture. As can be seen, each prosthesis utilizes an outer supply or extraction tube which can be coupled to a fluid supply or a vacuum as described above.

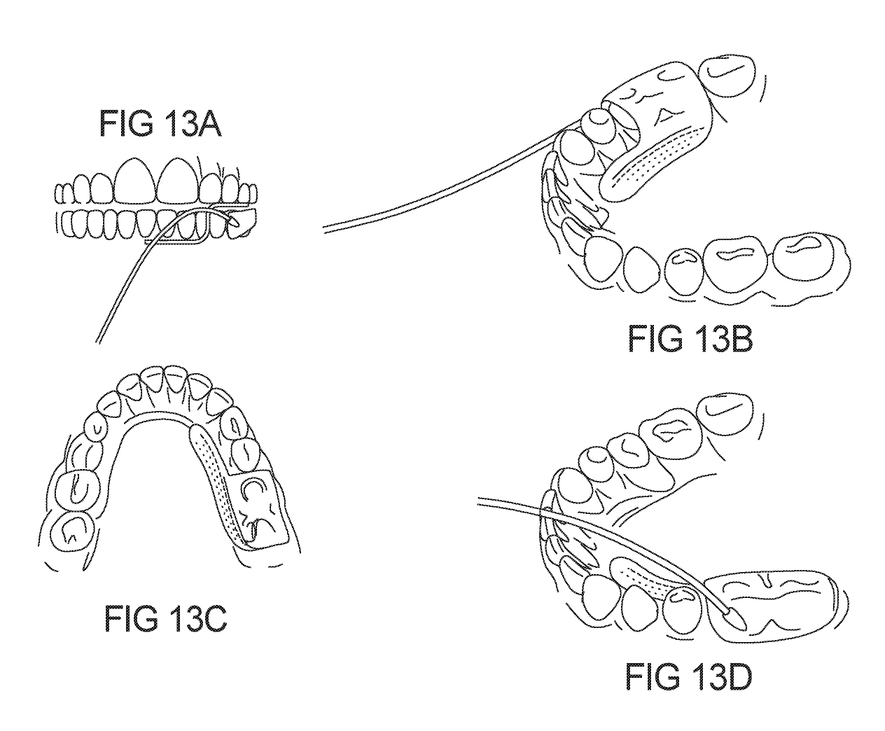

FIGS. 13a-13d represent over the tooth saliva replenishment prosthesis according to the present teachings. Similar to the teachings in FIGS. 7a-7d, alternate upper and lower saliva replenishment prosthesis according to the present teachings. As can be seen the prosthesis travels over the molar region and is positioned under the tongue at only a single location. The upper and lower prosthesis are made of materials having a soft durometer. They define an inner passage and a plurality of apertures which link an outer surface of the prostheses with the inner aperture. As can be seen, each prosthesis utilizes an outer supply or extraction tube which can be coupled to a fluid supply or a vacuum as described above.

In various aspects, the first and second anchoring members can be off-the-shelf components, semi-custom components, or custom components. As used herein, off-the-shelf components can refer to components made without features based on a particular user. Semi-custom components can refer to components made in advance that include a majority of predetermined features not based on a particular user and at least one feature based on a particular user. Custom components can refer to components specifically made for a particular user. The patient-specific features of a semi-custom component and a custom component can be formed based on a particular user's lower dental arch and surrounding mouth anatomy using various techniques such as dental impressioning techniques.

Each of the embodiments in FIGS. 7a-13d can utilize the foam member 100 can be Hydrasorb® Foam Hydrasorb® the name of a group of medical-grade polyurethane, hydrophilic foams. This foam is manufactured from a base material of polyether polyisocynate resins. Hydrasorb® is sterilizable. And can be Die cut or 18″×36″ sheets (⅛″ to ¾″ wetted thickness) or molded to shape. Hydrophilic Absorption Capacity (Water): up to 15×Dry Wt. [ASTM D1667] Cell Structure (DRY Avg.): 86 Cells/Linear In. Density (Nominal/DRY): 7.5 lb/ft3 [ASTM D3574] Elongation % (DRY Avg.): 650% [ASTM D3574] Expansion in Water (length)(Avg.): 31% (Avg.) [ASTM F1087] Foam Moisture Content (DRY Avg.): 3.56% [Karl Fischer Method] Indention Force Deflection (IDF): [ASTM D3574 Test B1] IDF @ 25%: (N) 133% IDF @ 65%: (N) 346% Resiliency/Rebound Test (RT [ASTM D3574 Test B1] RT @ 25%: (N) 121% Compression Set (DRY Avg.): [ASTM D3574] 25%: 16.0% 50%: 36.0% Tensile Strength (Dry): 30.0 lbf/In2 [ASTM D3574 Test B1]

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It is additionally envisioned the systems described above can be used in conjunction with a positive airflow sleep apnea machine. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A system for providing fluid to the mouth cavity of a user, the mouth cavity having a dental arch with a biting surface and vestibule on an outer side of the dental arch, and a gum line on an inner side of the dental arch, the system comprising:
a mouthpiece having a hydrophilic foam member configured to couple the mouthpiece to the dental arch, the mouthpiece having a first member configured to be adjacent the outer side of the dental arch, and a second member configured be annularly disposed about the hydrophilic foam member, the mouthpiece defining first fluid passage fluidly coupled the hydrophilic foam member;
a fluid conduit coupled to the first fluid passage; and
a supply source of vacuum coupled to the first fluid passage.

2. The system according to claim 1, wherein the mouthpiece comprises a plurality of anchoring members adapted to engage the dental arch.

3. The system according to claim 2, wherein the anchoring member is configured to engage the gum line.

4. The system according to claim 1, wherein the vacuum source comprises a pump and a controller configured to regulate the flow of saliva through the fluid conduit.
